(19) United States
(12) Patent Application Publication
(10) Pub. No.: US 2026/0063517 A1
(43) Pub. Date: Mar. 5, 2026

(54) METHOD FOR IDENTIFYING AND DETECTING TRACE POLLUTANTS IN SHIELD MUCK

(71) Applicants: SHENZHEN UNIVERSITY, Shenzhen (CN); China Construction Engineering (Hong Kong) Co., Ltd., HONG KONG (CN); China Construction Civil Engineering Co., Ltd., Hong Kong (CN)

(72) Inventors: ZIJUN DONG, Shenzhen (CN); MING ZHANG, HONG KONG (CN); GUO LYU, Shenzhen (CN); FOCI CHEN, Shenzhen (CN); XIANGSHENG CHEN, Shenzhen (CN); CHANGQING CHEN, Shenzhen (CN); XIAOHUI SUN, Shenzhen (CN); YUEFU ZHOU, Shenzhen (CN)

(21) Appl. No.: 19/333,803

(22) Filed: Sep. 19, 2025

Related U.S. Application Data
(63) Continuation of application No. PCT/CN2025/094184, filed on May 12, 2025.

(30) Foreign Application Priority Data
Aug. 29, 2024 (CN) 202411196440.9

(51) Int. Cl. G01N 1/30; G01N 21/31; G01N 33/24

(52) U.S. Cl. CPC G01N 1/30; G01N 21/31; G01N 33/24

(57) ABSTRACT

The present disclosure relates to the technical field of shield muck detection, specifically relating to a method for identifying and detecting trace pollutants in shield muck, including the following steps: Step S1. pre-treating shield muck; Step S2. extracting anionic surfactant; Step S3. removing interfering components; Step S4. detecting anionic surfactant; Step S5. calculating anionic surfactant content. The present disclosure directly uses fresh shield muck for extraction, avoiding the decomposition phenomenon of anionic surfactants caused by high-temperature drying in traditional methods, significantly improving the accuracy of detection results, and utilizing a laboratory oscillator to batch process 10-20 samples at once, simplifying operation steps, while reducing reagent consumption, and effectively avoiding the problem of gas leakage in separatory funnels, improving operational efficiency and safety of experimenters, enabling rapid and efficient completion of extraction of anionic surfactants in soil, having broad application prospects.

### CROSS-REFERENCE TO RELATED APPLICATIONS

The present application is a Continuation of PCT/CN2025/094184, filed on May 12, 2025, which claims priority to Chinese Patent Application No. 202411196440.9, filed on Aug. 29, 2024. The entire contents of the aforementioned applications are hereby incorporated by reference.

### TECHNICAL FIELD

The present disclosure relates to the technical field of shield muck detection, and particularly relates to a method for identifying and detecting trace pollutants in shield muck.

### BACKGROUND

Shield muck is formed by shield machines advancing in strata and cutting through cutterhead rotation, typically retaining large amounts of chemical additives injected into the strata to assist engineering excavation. At construction sites, shield muck is sieved into sand and gravel and silty mud cakes; for silty mud cakes requiring external transport to landfills, compared to normal farmland soil, they have smaller particle sizes and contain many chemical additives such as residual foaming agents from engineering excavation, exhibiting unique physical properties and complex chemical compositions. When chemically detecting specific pollutant components in shield muck, the treatment methods and detection differ from those for traditional soil chemical detection.

Currently, there is no standard method for determining multiple types of trace anionic surfactants in shield muck. Identification and detection of trace pollutants in shield muck mainly comprise extraction of pollutants from shield muck and detection of pollutants. The main pollutant in shield muck, the foaming agent, primarily consists of anionic surfactants, including substances such as sodium polyoxyethylene lauryl ether sulfate (AES), sodium dodecyl sulfate (SDS), and sodium lauryl ether sulfonate (SLES). For extraction of anionic surfactants in shield muck, no clear method exists yet. For detection of anionic surfactants, the commonly used method is the methylene blue spectrophotometric method. Traditional national standard methods involve complex operations, low analytical efficiency, and susceptibility to interference from various coexisting substances. For example, WAN Hanxing et al. published a study on the methylene blue method for total anionic surfactants in soil in “Environmental Science & Technology,” but fresh soil therein requires drying treatment at 105° C. High-temperature treatment causes decomposition of surfactants in the soil, leading to underestimated detection results. Additionally, reagent consumption per single detection in existing technologies is very high, and manual shaking during extraction using separatory funnels easily causes gas leakage, making it difficult to avoid volatilization of some reagents causing harm to experimenters.

Therefore, based on the related technologies mentioned above, there is an urgent need to develop a method for identifying and detecting trace pollutants in shield muck.

### SUMMARY

In view of this, an objective of the present disclosure is to propose a method for identifying and detecting trace pollutants in shield muck, aiming to provide a method capable of large-scale, rapid, and accurate detection of anionic surfactant content in shield muck.

Based on the aforementioned objective, provided in the present disclosure is a method for identifying and detecting trace pollutants in shield muck.

A method for identifying and detecting trace pollutants in shield muck including following steps:
Step S1. pre-treating shield muck;
Step S2. extracting anionic surfactant;
Step S3. removing interfering components;
Step S4. detecting anionic surfactant; and
Step S5. calculating anionic surfactant content.

The pre-treating shield muck in step S1 includes: naturally air-drying fresh soil of shield muck to be tested in a soil drying chamber for 24 to 48 hours, controlling moisture content at 10% to 20%, passing through a 10-mesh sieve, and mixing uniformly to obtain a shield muck soil sample, directly using fresh soil for extraction operation, avoiding the problem of inaccurate detection results caused by high-temperature treatment.

The extracting anionic surfactant in step S2 includes: placing the shield muck soil sample in a centrifuge tube, adding an extraction solution, oscillating in a horizontal oscillator, filtering the resulting mixture through a 5 μm needle filter after oscillation, and then performing water bath distillation to obtain a test solution.

In some implementations, a mass-volume ratio of the shield muck soil sample to the extraction solution is 1 g:20 mL.

In some implementations, the extraction solution is any one of pure water and an ethanol aqueous solution; a volume ratio of ethanol to water in the ethanol aqueous solution is (60-80):(40-50); and ethanol is removed during the water bath distillation process.

In some implementations, an oscillation frequency of the horizontal oscillator is 660 to 700 r/min; and an oscillation time is 8 to 10 min.

In some implementations, the specific operations of removing interfering components in step S3 comprises removing carboxylates, phenols, thiocyanates, cyanates, nitrates, and chlorides from the test solution.

In some implementations, the detecting in step S4 includes:
Step S41. placing an anionic surfactant standard solution in a centrifuge tube, adding a sodium hydroxide solution dropwise using phenolphthalein as an indicator until the solution turns peach red, then adding sulfuric acid dropwise until the peach red color just disappears, to obtain mixture 1;
Step S42. adding a methylene blue solution to mixture 1, fixing on a horizontal oscillator for oscillation, to obtain mixture 2;
Step S43. adding dichloromethane to mixture 2, fixing on a vertical oscillator for oscillation, and standing for layering after oscillation;
Step S44. aspirating the dichloromethane phase with a rubber-tipped dropper, injecting into a cuvette, and measuring absorbance of the system at a wavelength of 652 nm;
Step S45. placing the test solution in a centrifuge tube, adding a sodium hydroxide solution dropwise using phenolphthalein as an indicator until the solution turns peach red, then adding sulfuric acid dropwise until the peach red color just disappears, to obtain mixture 3;
Step S46. adding a methylene blue solution to mixture 3, fixing on a horizontal oscillator for oscillation, to obtain mixture 4;
Step S47. adding dichloromethane to mixture 4, fixing on a vertical oscillator for oscillation, and standing for layering after oscillation;
Step S48. aspirating the dichloromethane phase with a rubber-tipped dropper, injecting into a cuvette, and measuring absorbance of the system at a wavelength of 652 nm.

In some implementations, in step S41, an amount of the anionic surfactant is 5 mL, and a specification of the centrifuge tube is 15 mL.

In some implementations, in step S42, an amount of the methylene blue solution is 2 mL.

In some implementations, in step S42, a frequency of the horizontal oscillator is 660 to 700 r/min, and an oscillation time is 8 to 10 min.

In some implementations, in step S43, an amount of the dichloromethane is 5 mL.

In some implementations, in step S43, an oscillation frequency of the vertical oscillator is 600 to 700 r/min, and an oscillation time is 3 min.

In some implementations, in step S45, an amount of the test solution is 5 mL, and a specification of the centrifuge tube is 15 mL.

In some implementations, in step S46, an amount of the methylene blue solution is 2 mL.

In some implementations, in step S46, a frequency of the horizontal oscillator is 660 to 700 r/min, and an oscillation time is 8 to 10 min.

In some implementations, in step S47, an amount of the dichloromethane is 5 mL.

In some implementations, in step S47, an oscillation frequency of the vertical oscillator is 600 to 700 r/min, and an oscillation time is 3 min.

The calculating anionic surfactant content in step S5 includes:
Step S51. diluting the anionic surfactant standard solution with water, shaking uniformly, preparing into a plurality of anionic surfactant standard solutions with different mass concentrations, measuring absorbance values corresponding to the anionic surfactant standard solutions at different mass concentrations, plotting a standard curve with mass concentration of anionic surfactant as abscissa and a difference between measured absorbance value and absorbance value of zero-mass-concentration anionic surfactant standard solution as ordinate, and fitting a standard curve regression equation: y=ax+b, where x is anionic surfactant content, and y is absorbance;
Step S52. substituting the measured absorbance value of the test solution into the standard curve regression equation to calculate a mass concentration of anionic surfactant in the test solution.

In some implementations, the anionic surfactant includes sodium polyoxyethylene lauryl ether sulfate (AES), sodium dodecyl sulfate (SDS), sodium lauryl ether sulfonate (SLES), sodium alpha-olefin sulfonate (AOS), and sodium linear alkylbenzene sulfonate (LAS).

### Beneficial Effects of the Present Disclosure

1. Improving detection accuracy: Directly using fresh shield muck for extraction. Avoiding the decomposition phenomenon of anionic surfactants caused by high-temperature drying in traditional methods, thereby significantly improving the accuracy of detection results;
2. Optimizing extraction solution selection: Using pure water as the extraction solution, avoiding the influence of ethanol on the accuracy of dichloromethane extraction and the interference of ethanol's own absorbance, further improving the accuracy of detection results;
3. Reducing standing time: By immediately filtering the extraction solution using a 5 μm needle filter, effectively reducing standing time, avoiding the re-adsorption phenomenon of fine particles of muck on anionic surfactants, ensuring the purity of supernatant and the accuracy of subsequent detection;
Improving operational efficiency and safety: Using 15 mL capped centrifuge tubes combined with a vertical oscillator for vertical oscillation extraction, not only simplifying operation steps, utilizing laboratory oscillators to batch process 10-20 samples at once, reducing reagent consumption, but also avoiding the problem of gas leakage in separatory funnels, improving operational efficiency and safety of experimenters, enabling rapid and efficient completion of extraction of anionic surfactants in soil, with a recovery rate reaching 96.3%-104.5%.

### BRIEF DESCRIPTION OF THE DRAWINGS

In order to illustrate the technical solutions of the examples of the present disclosure or of the prior art more clearly, the following drawings are briefly described as required in the context of the examples or the prior art. Obviously, the following drawings illustrate only some of the examples of the present disclosure. Other relevant drawings may be obtained on the basis of these drawings without any creative effort by those skilled in the art.

FIG. 1 is a flowchart of the method for identifying and detecting trace pollutants in shield muck of the present disclosure;

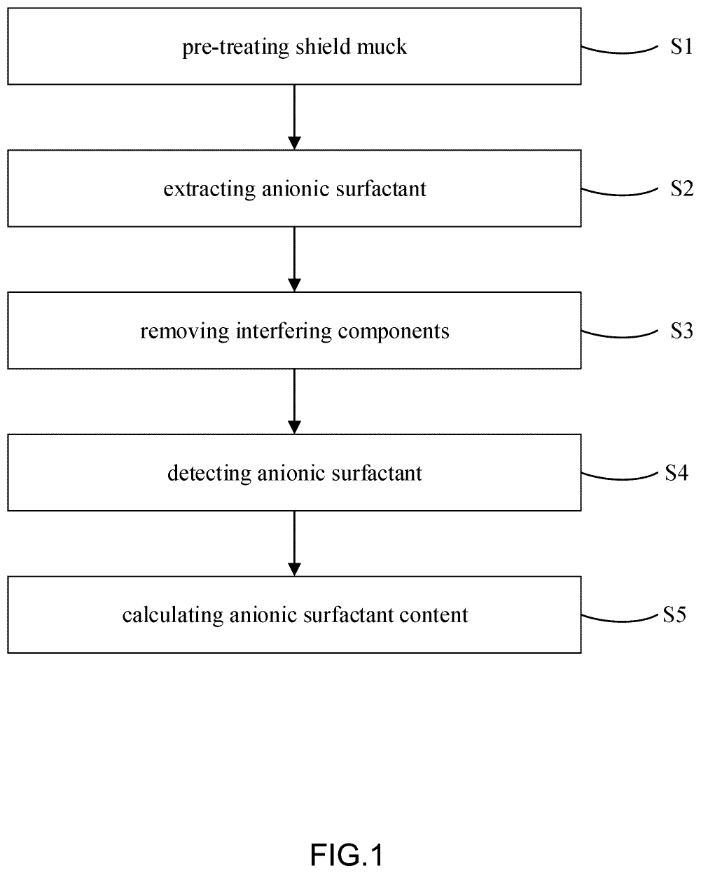

FIG. 2 is a mean and range quality control chart of absorbance for 20 sets of blank tests of the present disclosure;

FIG. 3 is a standard curve determined for sodium dodecyl sulfate (SDS) standard solution of the present disclosure;

FIG. 4 is a standard curve determined for sodium polyoxyethylene lauryl ether sulfate (AES) standard solution of the present disclosure;

FIG. 5 is a standard curve determined for sodium alpha-olefin sulfonate (AOS) standard solution of the present disclosure;

FIG. 6 is a standard curve determined for sodium linear alkylbenzene sulfonate (LAS) standard solution of the present disclosure.

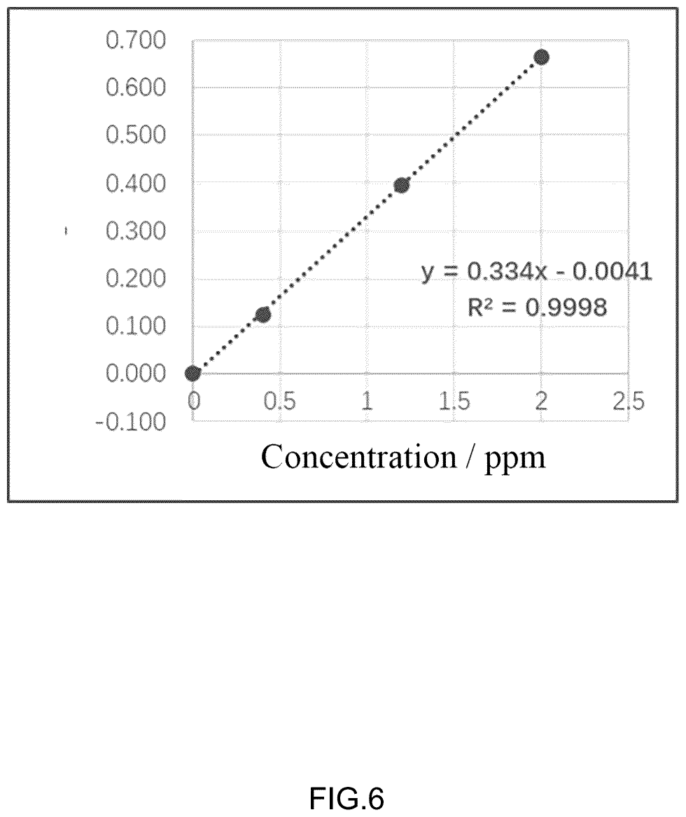

### DETAILED DESCRIPTION

To make the objectives, technical solutions, and advantages of the present disclosure clearer, the present disclosure is further described in detail below with reference to specific examples.

### Example 1: A Method for Identifying and Detecting Trace Pollutants in Shield Muck, Including the Following Steps

S1. Pre-treating shield muck: Fresh soil of shield muck to be tested was naturally air-dried in a soil drying chamber for 24 hours, with moisture content controlled at 10%. The soil was passed through a 10-mesh sieve and mixed uniformly to obtain a shield muck soil sample. Fresh soil was directly used for extraction operation, avoiding the problem of inaccurate detection results caused by high-temperature treatment. To make the soil sample more homogeneous and enable more complete extraction of analytes in subsequent steps, existing pretreatment methods generally prepared soil samples to be tested by sieving fresh soil through a 10-mesh sieve and mixing uniformly. However, it was difficult to perform fine sieving using methods for ordinary soil in its fresh state due to differences in physicochemical properties between shield muck and ordinary farmland soil, in which shield muck having finer particle size, higher moisture content, and higher viscosity. To address the issue of large variations in moisture content of shield muck from different sources and the difficulty of uniform sieving with conventional methods, the pretreatment method for shield muck from different sources was uniformly optimized. The soil sample to be tested was first naturally air-dried for 24 to 48 hours to control its moisture content at 10% to 20%, facilitating preparation. The primary purpose of air-drying was to reduce the high moisture content of shield muck to below 20%, decreasing soil adhesion, thereby facilitating subsequent sieving of large shield muck pieces through a 10-mesh sieve and reducing clogging and adhesion during sieving. After natural air-drying in the soil drying chamber for 24 hours, no decomposition of anionic surfactants in shield muck occurred because natural air-drying was mild and did not affect the thermal stability of surfactants. Natural air-drying was conducted at ambient temperature, and soil drying chambers typically simulated natural ventilation conditions with temperatures far below 105° C. Under such conditions, anionic surfactants exhibited high stability and were less prone to thermal decomposition. Anionic surfactants decomposed only under specific high-temperature conditions; for example, during high-temperature drying at 105° C., the molecular structure of surfactants might change due to rapid temperature increase, leading to decomposition.
S2. Extracting anionic surfactant: 1 g of the prepared soil sample was accurately weighed and placed in a 50 mL centrifuge tube (capped centrifuge tube). 20 mL of extraction solution was added to the centrifuge tube. The centrifuge tube was placed in a horizontal oscillator and oscillated at 700 r/min for 10 min. Immediately after oscillation, the extraction solution was filtered through a 5 μm needle filter to obtain 5 mL of test solution. Different shield foaming agents contained different anionic surfactant components, and their solubility in different extraction solutions varied. The selection of the pretreatment extraction solution was required to correspond to the main components of anionic surfactants in the actual shield muck. The extraction solutions included pure water and an ethanol aqueous solution: For shield muck dominated by sodium polyoxyethylene lauryl ether sulfate (AES) and sodium alpha-olefin sulfonate (AOS), which exhibits better solubility in pure water, pure water was directly used as the extraction solution. For shield muck dominated by sodium dodecyl sulfate (SDS), which exhibits stronger solubility in an ethanol aqueous solution, an aqueous solution with 60% ethanol content was selected as the extraction solution. Ethanol and water at this ratio provided excellent extraction efficiency for anionic surfactants. However, experiments found that while the use of an ethanol aqueous solution as the extraction solution for pretreatment of shield muck was feasible, subsequent methylene blue detection using the test solution containing ethanol resulted in higher absorbance in the blank group compared to that using pure water as the extraction solution. This was due to mutual solubility between ethanol and dichloromethane, and ethanol itself exhibited absorbance at 652 nm, causing interference in the detection of anionic surfactants and leading to inaccurate measurements. Therefore, to solve the problem of inaccurate results caused by ethanol in the extraction solution, pure water was adopted as the extraction solution in the present disclosure, or a method of distilling the ethanol aqueous solution test solution at 80° C. to remove ethanol was employed to avoid this issue. The spiked recovery rate measured by this method was closest to the spiked amount. This approach was based on the principle that ethanol has a lower boiling point than water; heating caused ethanol to evaporate, thereby achieving ethanol removal. During distillation, the distillation temperature (80° C.) and time were strictly controlled to ensure that sodium dodecyl sulfate (SDS) was not decomposed or lost due to high temperature. In the distillation process, ethanol evaporated preferentially owing to its lower boiling point, while SDS remained in the solution due to its higher boiling point. This distillation condition ensured effective removal of ethanol while minimizing loss of SDS. Experimental data also showed that after distillation, the recovery rate of SDS was very high, with no significant change in its component content. Typically, after oscillation extraction of muck with the extraction solution, the method to obtain the test supernatant involved standing for a certain period. However, in experiments using this approach, the detected spiked concentration was far lower than the actual value. Tests with standing times of 0, 10, and 20 minutes were conducted, and results revealed that the detected concentration of anionic surfactants in the supernatant decreased with longer standing time. This might be attributed to the adsorption of anionic surfactants by fine muck particles, causing dissolved surfactants to be re-adsorbed onto the particles and reducing their content in the final extraction solution. To immediately obtain the test supernatant after soil oscillation extraction, needle filter filtration was adopted in the present disclosure. Immediately after oscillation extraction, the extraction solution was filtered through a needle filter to obtain a clear solution. Among needle filters with pore sizes of 0.22 μm, 0.45 μm, and 5 μm, the 5 μm needle filter with higher filtration efficiency and effectiveness was selected to resolve this issue. Ultimately, the highest spiked recovery rate was obtained by the present disclosure.
S3. Removing interfering components: Shield muck has a complex chemical composition, which may contain other organic sulfates, sulfonates, carboxylates, phenols, and inorganic thiocyanates, cyanates, nitrates, and chlorides besides the main analyte. These substances may react with methylene blue to form blue complexes soluble in dichloromethane or chloroform, leading to overestimation of measurement results. Therefore, removal of these substances from the test solution is required. Specific methods to avoid interference from carboxylates, phenols, and inorganic thiocyanates, cyanates, nitrates, and chlorides are as follows: Positive interference (except for organic sulfates and sulfonates) may be eliminated by aqueous solution backwashing, where most interferences from chlorides and nitrates are removed. Aqueous solution backwashing primarily removes impurities and pollutants adhering to filter media or other surfaces through reverse-flowing water. Specific operations include: the test solution was placed in a reverse osmosis membrane pressure vessel; the test solution inside the vessel was flushed with clean water; the backwash valve was opened: the backwash valve of the equipment was activated to allow backwash water to flow reversely into the equipment from the outlet; water flow rate was controlled: the flow rate of backwash water was adjusted according to specific equipment conditions and requirements, typically being maintained at a moderate rate that is neither too fast nor too slow.
Step S4. detecting anionic surfactant: 5 mL of anionic surfactant standard solution was taken in a 15 mL centrifuge tube. Using phenolphthalein as an indicator, a sodium hydroxide solution was added dropwise until the solution turned peach red, followed by dropwise addition of sulfuric acid until the peach red color just disappeared to obtain mixture 1. 2 mL of methylene blue solution was added to mixture 1, and oscillation was performed on a horizontal oscillator at 700 r/min for 10 min to obtain mixture 2. 5 mL of dichloromethane was added to mixture 2, and oscillation was performed on a vertical oscillator at 700 r/min for 3 min. After oscillation, standing for layering was conducted. The dichloromethane phase was aspirated with a rubber-tipped dropper and injected into a cuvette, and absorbance of the system was measured at a wavelength of 652 nm. To address the problem in the prior art of inability to fully mix upper and lower layers of methylene blue solution uniformly, 15 mL capped centrifuge tubes were adopted to replace test tubes in the present disclosure. Vertical oscillation extraction of the solution to be extracted in the centrifuge tube was performed using a vertical oscillator, enabling more complete extraction. For test solutions of the same concentration, the highest detection value was obtained, and smaller variance in detection values was exhibited. Furthermore, reagent quantities from national standard methods were adjusted in the present disclosure, and 15 mL capped centrifuge tubes were employed as extraction containers. Detection validity could be ensured while leakage of toxic gases during oscillation extraction was avoided, and safety of experimental personnel was protected.
S5. 5 mL of the test solution was taken in a 15 mL centrifuge tube. Using phenolphthalein as an indicator, a sodium hydroxide solution was added dropwise until the solution turned peach red, followed by dropwise addition of sulfuric acid until the peach red color just disappeared to obtain mixture 3. 2 mL of methylene blue solution was added to mixture 3, and oscillation was performed on a horizontal oscillator at a frequency of 700 r/min for 10 min to obtain mixture 4. 5 mL of dichloromethane was added to mixture 4, and oscillation was performed on a vertical oscillator at a frequency of 700 r/min for 3 min. After oscillation, standing for layering was conducted. The dichloromethane phase was aspirated with a rubber-tipped dropper and injected into a cuvette, and absorbance of the system was measured at a wavelength of 652 nm.
S6. Calculating anionic surfactant content: The anionic surfactant standard solution was diluted with water, shaken uniformly, and prepared into a plurality of anionic surfactant standard solutions with different mass concentrations. Absorbance values corresponding to the anionic surfactant standard solutions at different mass concentrations were measured. A standard curve was plotted with mass concentration of anionic surfactant as abscissa and the difference between measured absorbance value and absorbance value of the zero-mass-concentration anionic surfactant standard solution as ordinate. A standard curve regression equation was fitted: y=ax+b, where x represents anionic surfactant content, and y represents absorbance. The measured absorbance value of the test solution was substituted into the standard curve regression equation to calculate the mass concentration of anionic surfactant in the test solution. Since foaming agent products used in shield construction are formulated by blending multiple anionic surfactants, detection of anionic surfactants in shield muck calculates the standard curve based on the predominant anionic surfactant in the actual foaming agent.

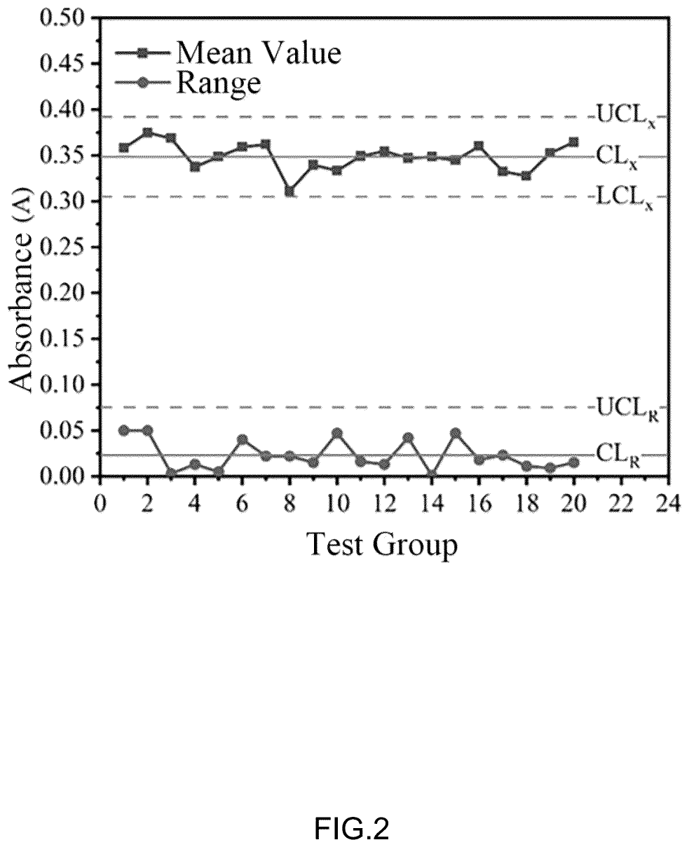

The mass concentration of anionic surfactant detected by the present disclosure met quality control requirements. As shown in the mean quality control chart of FIG. 2, absorbance values of 20 sets of blank tests all fell between UCLx and LCLx, with no seven consecutive points located on the same side of CLx or exhibiting consecutive increase/decrease, demonstrating stable system blanks that satisfied quality control requirements. In the range quality control chart of FIG. 2, absorbance ranges of 20 sets of blank tests all fell between UCLR and LCLR (where LCLR is a horizontal line with an ordinate of 0), with no seven consecutive points located on the same side of CLR or exhibiting consecutive increase/decrease, indicating that system errors were within control limits and quality control requirements were met.

### Standard Curve and Detection Limit:

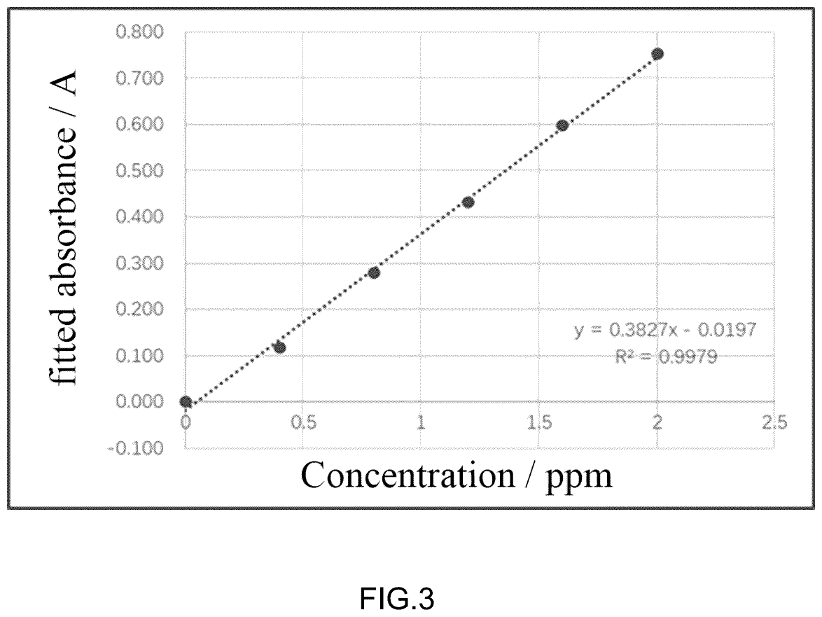

An appropriate amount of sodium dodecyl sulfate (SDS) standard solution was taken, diluted stepwise with water, shaken uniformly, and prepared into a series of standard solutions with SDS mass concentrations of 0, 0.4, 0.8, 1.2, 1.6, and 2.0 mg/L. Measurements were performed according to the test method. A standard curve was plotted with SDS mass concentration as abscissa and the difference between measured absorbance value and absorbance value of the 0 mg/L SDS standard solution as ordinate. As shown in FIG. 3, the linear range of the SDS standard curve was within 2.0 mg/L, with a linear regression equation of y=0.3827x−0.0197 and a correlation coefficient of 0.9979. According to regulations of the International Union of Pure and Applied Chemistry (IUPAC), the detection limit was calculated as three times the standard deviation(s) divided by the slope (k) of the linear regression equation (3s/k), resulting in 0.1947 mg/L.

Precision and recovery tests: Sodium dodecylbenzene sulfonate (LAS) standard solutions with mass concentrations of 0.2, 0.6, and 1.2 mg/L were measured six times each according to the test method of the present disclosure. Results showed that the relative standard deviations (RSD) of measured values are 1.150%, 1.219%, and 1.102% successively, meeting the requirement in the Manual for Quality Assurance of Environmental Water Monitoring (Second Edition) that intra-laboratory RSD should not exceed 20%, indicating high precision of the improved method.

For a sample with concentration of 0.4 mg/L, 0.5 mL, 1.75 mL, and 2.5 mL of 2.0 mg/L SDS standard solution was added to each group respectively, achieving spiked concentrations of 0.2 mg/L, 0.7 mg/L, and 1.0 mg/L. Recovery rates were 100.9%, 99.8%, and 103.3% successively, complying with the 95%-105% intra-laboratory spiked recovery rate requirement specified in the Manual for Quality Assurance of Environmental Water Monitoring (Second Edition), demonstrating high accuracy of the improved method.

Detection process for different anionic surfactant types: Standard solutions of sodium polyoxyethylene lauryl ether sulfate (AES), sodium alpha-olefin sulfonate (AOS), and sodium linear alkylbenzene sulfonate (LAS) with concentrations of 0, 0.4, 1.2, and 2.0 mg/L were prepared according to steps S1 to S3 of the present disclosure, with two parallel samples per group.

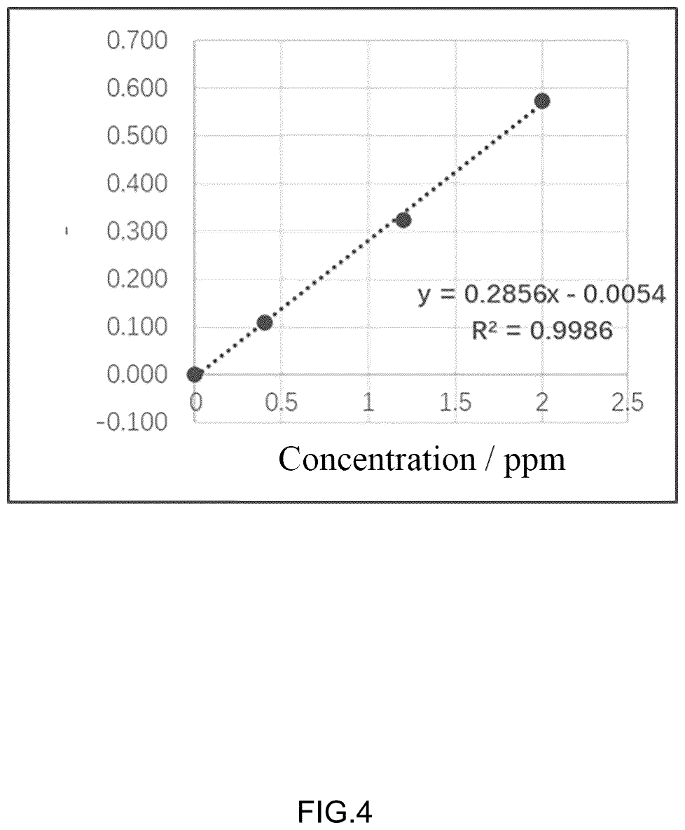

As shown in the results of FIG. 4, the linear range of the AES standard curve was within 2.0 mg/L, with a linear regression equation of y=0.2856x−0.0054 and a correlation coefficient of 0.9986.

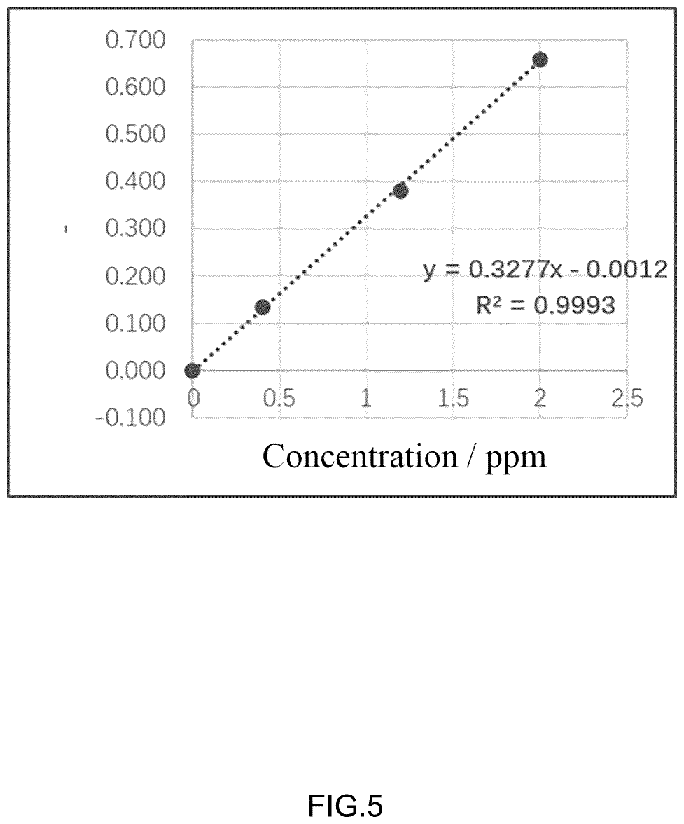

As shown in the results of FIG. 5, the linear range of the AOS standard curve was within 2.0 mg/L, with a linear regression equation of y=0.334x−0.0012 and a correlation coefficient of 0.9993. In the present disclosure, based on the ratio of sodium polyoxyethylene lauryl ether sulfate (AES), sodium alpha-olefin sulfonate (AOS), and sodium dodecyl sulfate (SDS) (4:4:2) in shield construction foaming agents, standard curves were fitted. The linear regression equation for AES (y=0.2856x−0.0054), AOS (y=0.334x−0.0041), and SDS (y=0.3827x−0.0197) were fitted into y=0.3244x−0.008.

TABLE 1
Differences In Physicochemical Properties Between Shield Muck And General Soil

| Physicochemical | | |
|---|---|---|
| Property | Shield Muck | General Farmland Soil |
| Source | Formed by sieving and filter | Naturally formed through |
| | pressing of slurry generated | long-term weathering and |
| | during shield construction | biological processes |
| Composition | Rich in clay minerals, residual | Minerals, organic matter, |
| | shield additives, etc. | microorganisms, etc. |
| Particle Size | Generally fine, mostly classified | Wide distribution including sand, |
| | as silt or clay particles | silt, and clay particles |
| Texture | Fine texture with higher | Varies depending on soil type |
| | viscosity | |
| Moisture Content | Typically 10%-50%, containing | Moderate moisture content |
| | higher water content | influenced by rainfall, irrigation, |
| | | etc. |
| Nutrient Content | Relatively low, requiring further | Varies based on soil type, |
| | treatment | fertilization, and cultivation |
| | | management |
| Environmental Impact | Contains residual shield | Typically environmentally |
| | additives and potentially harmful | friendly, supporting ecosystems |
| | substances, necessitating | |
| | environmental treatment | |

A person skilled in the art should understand that the discussion of any examples above is merely exemplary and is not intended to imply that the scope of the present disclosure is limited to these examples; under the concept of the present disclosure, technical features between the above examples or different examples may also be combined, steps may be implemented in any order, and there are many other variations in different aspects of the present disclosure as described above, which are not provided in detail for the sake of brevity.

The present disclosure aims to cover all such substitutions, modifications, and variations falling within the broad scope of the appended claims. Therefore, any omissions, modifications, equivalent substitutions, improvements, etc., made within the spirit and principle of the present disclosure, should be included within the protection scope of the present disclosure.

What is claimed is:

1. A method for identifying and detecting trace pollutants in shield muck, comprising the following steps:
Step S1. pre-treating shield muck;
Step S2. extracting anionic surfactant;
Step S3. removing interfering components;
Step S4. detecting anionic surfactant: measuring absorbance using methylene blue method; and
Step S5. calculating anionic surfactant content;
wherein the extracting anionic surfactant in step S2 comprises: placing the shield muck soil sample in a centrifuge tube, adding an extraction solution, oscillating in a horizontal oscillator, filtering the resulting mixture through a 5 μm needle filter after oscillation, and then performing water bath distillation to obtain a test solution;
the extraction solution is an ethanol aqueous solution.

2. The method for identifying and detecting trace pollutants in shield muck according to claim 1, wherein the pre-treating shield muck in step S1 comprises: naturally air-drying fresh soil of shield muck to be tested in a soil drying chamber for 24 to 48 hours, controlling moisture content at 10% to 20%, passing through a 10-mesh sieve, and mixing uniformly to obtain a shield muck soil sample.

3. The method for identifying and detecting trace pollutants in shield muck according to claim 1, wherein a mass-volume ratio of the shield muck soil sample to the extraction solution is 1 g:20 mL; a volume ratio of ethanol to water in the ethanol aqueous solution is (60-80):(40-50); an oscillation frequency of the horizontal oscillator is 660 to 700 r/min; and an oscillation time is 8 to 10 min.

4. The method for identifying and detecting trace pollutants in shield muck according to claim 1, wherein operations of removing interfering components in step S3 comprises removing carboxylates, phenols, thiocyanates, cyanates, nitrates, and chlorides from the test solution.

5. The method for identifying and detecting trace pollutants in shield muck according to claim 1, wherein the detecting in step S4 comprises:
Step S41. placing an anionic surfactant standard solution in a centrifuge tube, adding a sodium hydroxide solution dropwise using phenolphthalein as an indicator until the solution turns peach red, then adding sulfuric acid dropwise until the peach red color just disappears, to obtain mixture 1;
Step S42. adding a methylene blue solution to mixture 1, fixing on a horizontal oscillator for oscillation, to obtain mixture 2;
Step S43. adding dichloromethane to mixture 2, fixing on a vertical oscillator for oscillation, and standing for layering after oscillation;
Step S44. aspirating the dichloromethane phase with a rubber-tipped dropper, injecting into a cuvette, and measuring absorbance of the system at a wavelength of 652 nm;
Step S45. placing the test solution in a centrifuge tube, adding a sodium hydroxide solution dropwise using phenolphthalein as an indicator until the solution turns peach red, then adding sulfuric acid dropwise until the peach red color just disappears, to obtain mixture 3;
Step S46. adding a methylene blue solution to mixture 3, fixing on a horizontal oscillator for oscillation, to obtain mixture 4;
Step S47. adding dichloromethane to mixture 4, fixing on a vertical oscillator for oscillation, and standing for layering after oscillation; and
Step S48. aspirating the dichloromethane phase with a rubber-tipped dropper, injecting into a cuvette, and measuring absorbance of the system at a wavelength of 652 nm.

6. The method for identifying and detecting trace pollutants in shield muck according to claim 5, wherein in step S41, an amount of the anionic surfactant is 5 mL, and a specification of the centrifuge tube is 15 mL.

7. The method for identifying and detecting trace pollutants in shield muck according to claim 5, wherein in step S42, an amount of the methylene blue solution is 2 mL;
in step S42, a frequency of the horizontal oscillator is 700 r/min, and an oscillation time is 10 min;
in step S43, an amount of the dichloromethane is 5 mL;
in step S43, an oscillation frequency of the vertical oscillator is 700 r/min, and an oscillation time is 3 min.

8. The method for identifying and detecting trace pollutants in shield muck according to claim 5, wherein in step S45, an amount of the test solution is 5 mL, and a specification of the centrifuge tube is 15 mL;
in step S46, an amount of the methylene blue solution is 2 mL;
in step S46, a frequency of the horizontal oscillator is 660 to 700 r/min, and an oscillation time is 10 min;
in step S47, an amount of the dichloromethane is 5 mL; in step S47, an oscillation frequency of the vertical oscillator is 660 to 700 r/min, and an oscillation time is 3 min.

9. The method for identifying and detecting trace pollutants in shield muck according to claim 1, wherein the calculating anionic surfactant content in step S5 comprises:
Step S51. diluting the anionic surfactant standard solution with water, shaking uniformly, preparing into a plurality of anionic surfactant standard solutions with different mass concentrations, measuring absorbance values corresponding to the anionic surfactant standard solutions at different mass concentrations, plotting a standard curve with mass concentration of anionic surfactant as abscissa and a difference between measured absorbance value and absorbance value of zero-mass-concentration anionic surfactant standard solution as ordinate, and fitting a standard curve regression equation: y=ax+b, wherein x is anionic surfactant content, and y is absorbance;
Step S52. substituting the measured absorbance value of the test solution into the standard curve regression equation to calculate a mass concentration of anionic surfactant in the test solution;
wherein the anionic surfactant is any one of sodium polyoxyethylene lauryl ether sulfate (AES), sodium dodecyl sulfate (SDS), sodium lauryl ether sulfonate (SLES), sodium alpha-olefin sulfonate (AOS), and sodium linear alkylbenzene sulfonate (LAS).
